(19) United States
(12) Patent Application Publication
(10) Pub. No.: US 2003/0032825 A1
(43) Pub. Date: Feb. 13, 2003

(54) METHOD FOR PURIFYING AZELAIC ACID

(76) Inventors: DENNIS G GAIGE, FAIRFIELD, OH (US); KENNETH R MCVAY, HAMILTON, OH (US); EDWARD L EWBANK, FAIRFIELD, OH (US)

Correspondence Address: JOHN E. DRACH, COGINIS CORPORATION PATENT LAW DEPARTMENT, 2500 RENAISSANCE BOULEVARD, SUITE 200, GULPH MILLS, PA, 19406, US

(21) Appl. No.: 09/106,592

(22) Filed: Jun. 29, 1998

Related U.S. Application Data
(63) Continuation of application No. 08/451,290, filed on May 26, 1995, now abandoned.

(51) Int. Cl. C07C 59/185

(52) U.S. Cl. 554/121

(57) ABSTRACT

The invention is a method for recovering azelaic acid from a mixed oxidation product by prepurifying the azelaic acid and introducing the prepurified azelaic acid into the central portion of a dual countercurrent extraction column operated at an elevated temperature and introducing an aqueous phase into the top of the extraction column and a water immiscible solvent for the monocarboxylic acids in the prepurified azelaic acid into the bottom portion of the extraction column and recovering the purified azelaic acid from the aqueous phase. The azelaic acid can be recovered from the aqueous phase by drying the aqueous phase and separating a low boiling fraction and a high boiling fraction from the azelaic acid product by distillation.

### FIELD OF THE INVENTION

The invention is a process for purification of dicarboxylic acids formed by oxidation of unsaturated fatty acids. More particularly, the invention is a process for purification of azelaic acid formed by ozonation of unsaturated fatty acids. In particular, the present invention is directed to the final purification of azelaic acid formed by ozonization and oxidation of fatty acid mixtures containing oleic acid.

### BACKGROUND OF THE INVENTION

The specifications for commercial and polymer grade azelaic acid require a high percentage of azelaic acid and low amounts of mono-carboxylic acids in the material. It is known that when azelaic acid is produced by ozonization or oxidation of mixtures of fatty acids containing oleic acid, the oxidation product contains substantial amounts of carboxylic acids other than azelaic acid. On a molecular basis one mole of pelargonic acid is produced for each mole of azelaic acid produced. Other mono- and dicarboxylic acids are obtained by the ozonization or oxidation of carboxylic acids having more than one double bond or isomers of oleic acid in which the double bond has been displaced. In addition, since oleic acid is generally obtained from natural fats and oils, the oleic acid is contaminated with saturated fatty acids containing from about 12 to about 20 carbon atoms. The oxidation mixture from the ozonization process also contains monocarboxylic acids having from about 1 to about 8 carbon atoms and hydrocarbon materials. The nature of the constituents in the mixture and the overlap in boiling points of the various materials makes it difficult to recover a commercial grade (above about 90% by weight) azelaic acid containing substantially no monocarboxylic acid (less than 0.5%) from the mixture.

### RELATED ART

U.S. Pat. No. 2,450,858 discloses a process for production of azelaic acid by the oxidation of oleic acid with an aqueous chromic acid/sulfuric acid mixture. The sulfuric acid/chromic acid materials are washed from the reaction product. Azelaic acid is recovered from the reaction product by a first topping distillation which separates the pelargonic acid and lower boiling point materials from the mixture. The remaining material containing azelaic acid is contacted with several portions of water to produce an aqueous solution of azelaic acid which contains not more than 10% by weight of azelaic acid. The '858 patent teaches that it is important to maintain the concentration of azelaic acid in the aqueous phase below 10% by weight since as the concentration of azelaic acid increases above 10% by weight the solution acts as a solvent for water insoluble materials. After the extraction with water, the water phase is separated from the non-water soluble material and the azelaic acid recovered. The azelaic acid can be recovered from the water solution by a crystallization process or by evaporation of the water from the solution. Preferred method is by evaporation of the water from the solution.

It is not possible by the method disclosed in U.S. Pat. No. 2,450,858 to produce an azelaic acid product containing more than 90% by weight azelaic acid and less than about 0.5% by weight of monocarboxylic acids without additional processing or a substantial loss in azelaic acid during the crystallization step.

U.S. Pat. No. 2,813,113 (which is incorporated herein by reference) discloses a process for the preparation of azelaic acid by ozonization and oxidation of a mixture of acids containing oleic acid. The patent is directed to a method of ozonization and oxidation of the ozonide formed.

In the process disclosed in U.S. Pat. No. 2,813,118 a mixture of acids containing as a major constituent pelargonic acid is added to the oleic acid mixture to reduce the viscosity of the ozonide produced. The azelaic acid is recovered from the mixed oxidation products by removing a pelargonic acid fraction from the mixture by a distillation step. Pelargonic acid produced by the process is more than 40% by weight of the oxidation products and the pelargonic acid recycled to reduce the viscosity of the ozonide brings the total amount of pelargonic acid in the mixed oxidation products to the range of about 60% or higher. After removal of the pelargonic acid and lower boiling components the mixture is then passed to a distillation column wherein the azelaic acid and closely boiling materials are separated from the tars and pitches formed in the ozonization and oxidation process. The mixed acids taken overhead as a distillate from the second distillation column contain the azelaic acid, and other mono- and dicarboxylic acids which have boiling points from about the boiling point of pelargonic acid to below the boiling point of the tars and pitch removed in the bottoms product from the second distillation column.

The mixed acids from the overhead of the second distillation step are dissolved in hot water and the water soluble azelaic acid and other water soluble materials are separated from the non-water soluble monocarboxylic acids. The azelaic acid is then recovered from the solution by crystallization or by removal of the water from the solution. This purification process does not produce azelaic acid containing more than 90% azelaic acid without a substantial loss in azelaic acid.

U.S. Pat. No. 2,916,502 discloses a process for separating monocarboxylic from dicarboxylic acids. The process comprises dissolving the mixed acids in an aqueous aliphatic polyalcohol or an aqueous monocarboxylic acid ester of the polyalcohol then contacting the solution with a petroleum distillate having a boiling point from 30° C. to 100° C. The dicarboxylic acid is recovered from the aqueous solvent, separation of a mixture of azelaic and pelargonic acid is exemplified.

U.S. Pat. No. 2,998,439 discloses a process for recovering the azelaic acid from an oxidation reaction product. In the process, the oxidation reaction product acid is dissolved in at least five times its weight of water and the solution is counter currently contacted at a temperature above 85° C. with a non-polar solvent for the non-water soluble materials. The extracted solution of the azelaic acid is crystallized to recover the azelaic acid. The non-polar solvent-phase is evaporated to recover the non-polar solvent and the monocarboxylic acids.

The process as disclosed separates the azelaic acid from the total oxidation product without a prior separation. Generally, this means that the azelaic acid is present in the aqueous phase of the extraction step in relatively small amounts. The process is not capable of producing an azelaic acid product containing more than 90% by weight of azelaic acid and less than 1% by weight of monocarboxylic acids from a mixed oxidation product without substantial loss of azelaic acid.

U.S. Pat. No. 3,402,108 discloses a process for the production of a purified grade of azelaic acid. U.S. Pat. No. 3,402,108 is incorporated herein by reference. U.S. Pat. No. 3,402,108 discloses preparation of a mixed oxidation product containing azelaic acid and a method of recovering a purified azelaic acid from the mixed oxidation product. The process for recovering the azelaic acid comprises separating the short chain length carboxylic acids from the mixture by a first distillation step, the azelaic acid being in the under-flow from the column. The azelaic acid and the other carboxylic acids boiling in the range of azelaic acid are separated from the high boiling point residues in a second distillation column. The azelaic acid containing monocarboxylic acids and dicarboxylic acids is taken overhead from the distillation column and passed to an extraction zone wherein the azelaic acid mixture is contacted with hot water to form an aqueous solution of azelaic acid and separated from the non-soluble C₁₀-C₁₈ monocarboxylic acids. The aqueous solution of the azelaic acid is then contacted with an oxygen-ozone mixture in a bleaching zone and the bleached aqueous azelaic acid containing solution is counter currently contacted with a non-polar hydrocarbon material such as octane to reduce the amount of monocarboxylic acids in the azelaic acid solution. The azelaic acid solution is then crystallized and filtered to separate the azelaic acid crystals from the aqueous phase. The azelaic acid crystals are then dried and molten azelaic acid is distilled to separate a purified azelaic acid from a high boiling point residue. The patent exemplifies preparation of an azelaic acid product containing about 92% by weight azelaic acid and less than 0.04% by weight of monocarboxylic acids. The product was prepared from a mixture formed by oxidation of an acid mixture containing 73.5% by weight of oleic acid, 10% by weight of C₁₄-C₁₈ monocarboxylic acids and about 7.5% by weight of other mono-unsaturated fatty acids and 9% by weight of poly-unsaturated fatty acids. The reference teaches that the ozonization of the azelaic acid solution is necessary to obtain an azelaic acid product containing more than 90% by weight of azelaic acid with good color and color stability.

Applicants have unexpectedly discovered a process by which azelaic acid can be recovered from a mixed oxidation product. The product contains more than 90% by weight of azelaic acid and less than 0.05% by weight of monocarboxylic acids.

### BRIEF DESCRIPTION OF THE INVENTION

The process of the present invention includes separation of an azelaic acid mixture (hereinafter prepurified azelaic acid) from the major portion of pelargonic acid formed in the process and lower boiling point carboxylic acids and higher boiling point residue to form the prepurified azelaic acid mixture, feeding the prepurified azelaic acid mixture into a central portion of a dual extraction zone wherein the prepurified azelaic acid is continuously contacted with an aqueous phase and a water immiscible solvent phase to form an aqueous phase containing the azelaic acid and water soluble carboxylic acids and a water immiscible solvent phase containing the monocarboxylic acids and a portion of the dicarboxylic acids having a chain-length of 9 or greater and recovering the azelaic acid from the aqueous phase.

The azelaic acid can be recovered from the aqueous phase by removing the water from the aqueous phase and distilling the extracted azelaic acid to remove a fraction with a boiling point lower than azelaic acid and introducing the azelaic acid into a second distillation column to recover the azelaic acid as an overhead product and a residue comprising dicarboxylic acids, said residues having boiling points higher than azelaic acid.

The distillation operation can be controlled to provide an azelaic acid containing more than 90% azelaic acid by controlling the amount of carboxylic acids with boiling points higher than azelaic acid which are removed from the azelaic acid in the final distillation step.

In an alternate procedure, the azelaic acid can be recovered from the aqueous phase by a crystallization and the water phase recycled to the extraction column as a portion of the aqueous phase, the crystallized azelaic acid can be melted and passed to a distillation zone wherein the purified azelaic acid is recovered as an overhead product from a single distillation step or can be recovered by a two distillation step process.

As an alternate process, the prepurified azelaic acid before entering the dual extraction zone can be subjected to an additional distillation step which removes an overhead lighter fraction and a high boiling point bottoms residue and a further purified azelaic acid product is removed from the distillation column above the residue and below the feed point to the column and introducing the azelaic acid into the central portion of a dual extraction column utilizing the water and water immiscible solvent for the monocarboxylic acids, and recovering the azelaic acid from the aqueous phase by evaporation of the water and recovering the azelaic acid in a single step or two step distillation as an overhead product.

### BRIEF DESCRIPTION OF THE DRAWINGS

FIG. 1 is a schematic representation of the extraction process of the invention.

FIG. 2 is a schematic diagram of an azelaic acid recovery process utilizing the dual extraction column and a crystallization procedure with recycle of the aqueous phase for recovery of the azelaic acid.

FIG. 3 is a schematic representation of a process wherein the azelaic acid is recovered by evaporation of the aqueous phase and a distillation procedure.

FIG. 4 is a schematic representation of a process in which a prepurified azelaic acid is distilled to provide an additionally purified azelaic acid which is then extracted in the dual extraction column with an aqueous phase and the water immiscible solvent for the monocarboxylic acids, the aqueous phase dried and the azelaic acid recovered in a single step distillation.

### DETAILED DESCRIPTION OF THE INVENTION

As used in the following description, all percentages are percent by weight unless otherwise noted.

In the process of the invention, the mixed oxidation products from the oxidation step, in the preparation of azelaic acid, is prepurified by a distillation procedure to provide a prepurified azelaic acid feed to a dual extractor. Prepurification of the mixed oxidation products is necessary since more than 50% by weight of the oxidation product is pelargonic acid which is produced by the ozonization and oxidation and in addition a portion of which is recycled to reduce the viscosity of the ozonide during the ozonization procedure.

The distillation prepurification removes components such as hydrocarbons, monocarboxylic acids and dicarboxylic acids with boiling points lower than azelaic acid and in addition removes compounds with boiling points higher than azelaic acid. In the prepurification step, from about 40 to 60% by weight of the mixed oxidation products are separated from a crude azelaic acid material.

The distillation procedure by which a portion of the lower boiling point and higher boiling point materials are separated from the azelaic acid are multi-component distillations which are difficult to carry out to provide a pure azelaic acid. This is particularly true since many of the dicarboxylic acids and monocarboxylic acids have boiling points close to the boiling point of azelaic acid. Distillation recovery of azelaic acid on a commercial scale containing substantially no monocarboxylic acid, from a mixed oxidation product, is not possible.

In the process of the invention, the prepurified azelaic acid is introduced into the central portion of a dual extraction apparatus. The aqueous phase is introduced into one end of the extraction apparatus and a water immiscible solvent for the monocarboxylic acid is introduced into the opposite end of the extraction apparatus. Since the water immiscible solvent is generally a non-polar hydrocarbon material with a specific gravity lower than water, it is introduced into the bottom portion of the extraction column. The operation of the extractor will be described in relation to use of a water immiscible solvent for the monocarboxylic acids having a specific gravity lower than that of the aqueous phase. However, it must be realized that it is possible to use water immiscible solvents having specific gravities higher than the aqueous phase.

In the upper portion of the extraction apparatus utilized in the practice of the present invention, the rising water immiscible solvent for the monocarboxylic acids is contacted with a water phase to reduce the amount of azelaic acid and other dicarboxylic acids in the water immiscible solvent phase. In the central portion of the apparatus, the azelaic acid is mixed and dissolved in contact with the aqueous phase and the solvent phase In the lower portion of the extraction apparatus, the aqueous phase is extracted with the water immiscible solvent for the monocarboxylic acid. In the lower portion of the column, the monocarboxylic acids are substantially removed from the aqueous phase to provide an aqueous phase in which the non-volatile portion contains less than 0.5% by weight of monocarboxylic acids and preferably less than 0.1% by weight of monocarboxylic acids and most preferably less than 0.05% by weight monocarboxylic acids.

The extractor column is of a multi-stage design. Preferably a design such as a York-Scheibel or a Treybal column which provides multiple stage agitation and contact between the aqueous and the water immiscible phases.

In the process of the invention, the column is operated at an elevated temperature in the range of from about 65° C. to about 95° C. preferably in the range of 70° C. to 90° C. and most preferably in the range of 72° C. to 87° C. The elevated temperature is useful for providing a more concentrated solution of the azelaic acid and solutions with sufficiently low viscosities that are readily separated in the stages of the extractor apparatus. The extraction can be carried out at temperatures above 100° C. but pressure equipment is required and little advantage is achieved at temperatures above 100° C. In operation of the apparatus, the ratio of aqueous phase to prepurified azelaic acid is preferably in the range of from about 3:1 to about 9.0:1. The ratio of the aqueous phase to the prepurified azelaic acid is dependant on the amount of azelaic acid in the prepurified azelaic acid. It is preferred that the azelaic acid solution which exits the bottom of the column contains from about 10.5 to about 18% by weight of azelaic acid and most preferably from about 11.5 to about 15%. The ratio of aqueous phase to the prepurified azelaic acid is dependant upon the quantity of azelaic acid in the prepurified azelaic acid entering the dual extraction apparatus.

The weight ratio of the aqueous phase to the water immiscible solvent phase is from about 3.5:1 to about 20:1 when the water immiscible solvent has a specific gravity in the range of about 0.8 to about 0.9 and preferably a ratio of about 4:1 to about 16:1.

The water immiscible solvent, utilized in the practice of the present invention, is a solvent for monocarboxylic acids, is substantially immiscible in the aqueous phase, is a poor solvent for azelaic acid and has a sufficiently low boiling point that it can be readily separated from the materials extracted from the prepurified azelaic acid for recycle to the extraction zone. In addition, the boiling point should be sufficiently high that the extraction apparatus can be operated at substantially atmospheric pressure. In addition, the boiling point of the solvent should be sufficiently high that the danger of explosions and fires is substantially reduced. Aliphatic hydrocarbon solvents and mixtures of aliphatic hydrocarbons are particularly useful since they are solvents for the monocarboxylic acids and can be readily recovered at reasonable temperatures from the monocarboxylic acids. Solvents with boiling points up to about 200° C. which are water immiscible and good solvents for the monocarboxylic acids are preferred. Solvents with boiling points above about 90° C. are also preferred.

The extracting zone or device utilized in the practice of the invention, to carry out the extraction, can be any counter current extracting device which provides intimate contact between a rising and a descending phase. Moreover, since the prepurified azelaic acid is introduced directly into the center portion of the column, mixing means must be provided to adequately disperse the prepurified azelaic acid into the aqueous phase and the water immiscible solvent phase. In the prior art processes the azelaic acid to be extracted, was dissolved in water, insoluble materials separated and the water phase containing the dissolved azelaic acid was then contacted with a water immiscible solvent. In the present invention, prepurified azelaic acid is dissolved in the aqueous phase which is in contact with the water immiscible solvent phase. The prepurified azelaic acid is introduced into the extractor in the central portion and contacted with both the water phase and the water immiscible solvent phase.

As the aqueous phase containing the dissolved azelaic acid proceeds down the column through the extraction stages, the water insoluble materials are extracted from the aqueous phase and pass into the water immiscible solvent phase. In the dual extraction apparatus utilized in the practice of the present invention, the water immiscible solvent phase is extracted with an aqueous phase in the upper portion of the column, to reduce the content of water soluble materials in the water immiscible solvent phase, in the section of the extractor above the point at which the prepurified azelaic acid is introduced.

In the extractor below the point at which the prepurified azelaic acid is introduced into the apparatus, the water immiscible solvent phase extracts the water insoluble materials from the aqueous phase. In the process, the amount of monocarboxylic acids in the azelaic acid is substantially reduced and in particular is reduced to concentrations below 0.5% and preferably below about 0.1% and most preferably below about 0.05% of the amount of azelaic acid dissolved in the aqueous phase. The amount of water soluble material in the solvent phase is reduced by contact with the aqueous phase in the portion of the extractor column above the point at which the prepurified azelaic acid is introduced into the column.

Another advantage of the extraction apparatus of the present invention resides in the fact that if the azelaic acid is recovered by crystallization from the aqueous phase, a portion of the aqueous phase can be recycled to replace a portion of the aqueous solvent entering the extraction apparatus. The ability to recycle the aqueous phase from which the azelaic acid has been crystallized increases the yield of azelaic acid and reduces the amount of aqueous phase which must be removed from the process and treated.

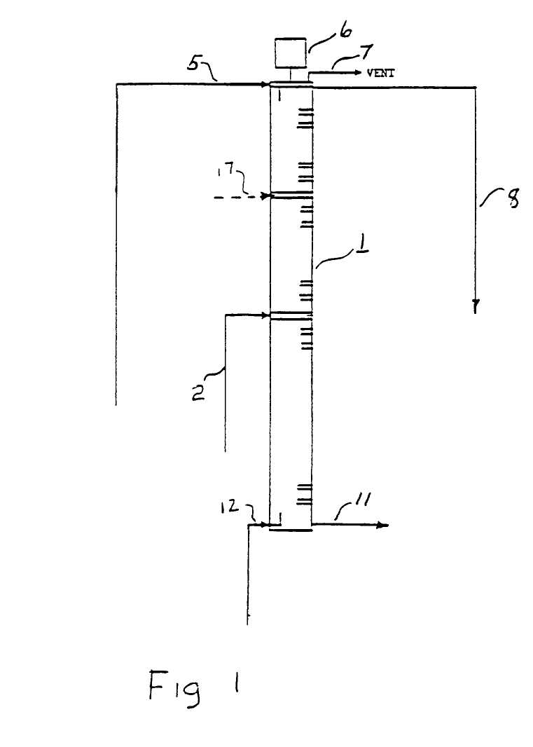

FIG. 1 is a schematic representation of a dual extractor apparatus which is useful in the practice of the invention. The extractor column exemplified is of a York-Scheibel type having alternating mesh packing and open spaces. The open spaces between the sections of mesh packing contain rotating mixing means to intimately mix the aqueous phase with the water immiscible solvent. The extractor column generally has 30 to 100 or more of the packed sections with open spaces in between containing the rotating mixing means.

The molten prepurified azelaic acid enters the central portion of the extractor through line 2 and is mixed with the aqueous phase and the water immiscible solvent phase in the column. Heated water enters near the top of the column through line 5. A quiescent separation zone in which the water immiscible solvent phase is separated from the aqueous phase is provided at the top of the extractor. Entering water is heated to a temperature in the range of about 65 to about 95° C. by heat exchange means (not shown). Heated water immiscible solvent enters extractor 1 through line 12. As shown, the water immiscible solvent enters at a point above the bottom of the column to provide a quiescent zone for separation of the water immiscible solvent from the aqueous phase. The aqueous phase is removed from the extractor column through line 11. The water immiscible solvent phase leaves the extractor column 1 through line 8 and is treated in a solvent recovery zone (not shown) in which the water immiscible solvent is separated from the materials extracted from the azelaic acid, heated and returned to the column through line 12. A vent 7 is provided to maintain the proper pressure in the column. A rotating means 6 is provided to rotate the mixing means in the column.

If the azelaic acid is recovered from the aqueous phase by a crystallization step, a portion of the aqueous phase can be recycled to the extractor and enters the column through line 17 (shown as dotted line) at an upper portion below the point at which the make-up water is introduced into the extractor and above the point at which prepurified azelaic acid is introduced into the extractor. Introduction of the recycle aqueous phase below the point at which the make-up water is introduced into the extractor, permits contact of the water immiscible solvent with an aqueous phase which does not contain any azelaic acid or at most low concentration of azelaic acid and provides for a maximum driving force to reduce the azelaic acid concentration in the water immiscible solvent phase.

The ability to recycle a portion of the aqueous phase from the crystallization step, in which the azelaic acid is separated from the aqueous phase, reduces the volume of aqueous phase which must be treated to recover dissolved materials and/or disposed of. The introduction of the recycle stream into the extractor in line 17 is shown as a dotted line since it is not necessary to recover the azelaic acid by a crystallization step but provides an optional method if a more pure azelaic acid product is required.

Applicants have unexpectedly discovered that by utilizing the novel extraction process of the invention, commercial azelaic acid can be provided merely by removing the water from the aqueous phase, drying the azelaic acid and subjecting the azelaic acid to a two-step distillation procedure.

The two-step distillation procedure is required only if the purity of the azelaic acid must be above 90% by weight. The pre-purification is designed to reduce the amount of low boiling point mono- and dicarboxylic acids to acceptable levels and to reduce the amount of material entering the extraction zone. The extraction step substantially eliminates the azelaic acid soluble monocarboxylic acids from the mixture and high purity azelaic acid can be produced by removing the water from the aqueous phase followed by a single step distillation; i.e., a distillation step in which the azelaic acid is the overhead product and higher boiling point materials which generally include the color bodies are removed from the bottom of a distillation. apparatus. The process of the present invention provides a low color, color stable azelaic acid without need for ozonization of the aqueous phase as shown in U.S. Pat. No. 3,402,108.

The mixed oxidation products contain azelaic acid and by-product acids which are removed from the azelaic acid during processing. The by-product acids are predominantly mono- and dicarboxylic acids with chain lengths in the range of from about 2 to about 20 carbon atoms. As one skilled in the art understands, in distilling these materials, to prevent thermal damage, the distillations are generally carried out at reduced pressure, in the range of about 1 to about 40 mm Hg, so that the vapor can be formed in the bottom of the column without undue thermal stress on the products. One skilled in the fatty chemical art, would know pressures and temperatures at which the distillation separations can be made to prevent thermal degradation of the product. Temperature and pressure for operation of the distillation column is dependant upon the boiling point of the materials to be separated and their sensitivity to elevated temperatures. Generally temperatures up to about 280° C. are used at pressures of from 1.0 to 30 mm Hg.

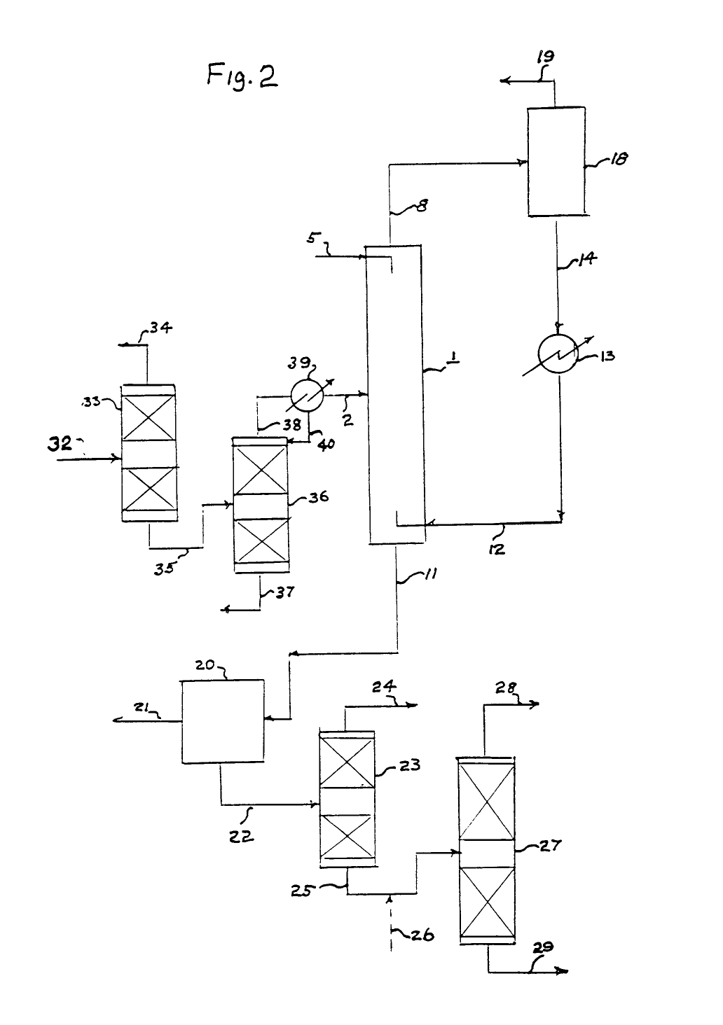

FIG. 2 is a schematic representation of a process which treats the mixed oxidation products through the production of purified azelaic acid. The same numbers are used when referring to the same apparatus as in FIG. 1.

FIG. 2 is a schematic representation of a process for separating azelaic acid from mixed oxidation products in a process in which the azelaic acid is formed by oxidation of an unsaturated carboxylic acid preferably oleic acid. As one skilled in the art understands, the process can also be applied to recovery of a dicarboxylic acid other than azelaic acid from a reaction mixture formed by oxidation of an unsaturated carboxylic acid other than oleic acid. The mixed oxidation products can be formed by processes in which the unsaturated carboxylic acid is oxidized with mixtures of materials such as sulfuric and chromic acids or formation of ozonides and oxidation of the ozonides.

As shown in FIG. 2, the mixed oxidation products enter distillation apparatus 33 through line 32. A major portion of the materials with a boiling point lower than the boiling point of azelaic acid are removed from the distillation apparatus through line 34. A major portion of the mixed oxidation product entering through line 32 is removed through line 34 and comprises pelargonic acid if the starting material was oleic acid. Distillation apparatus for fatty acid products are known and are generally operated at reduced pressure to minimize thermal degradation of the materials. As shown the distillation columns are preferably packed columns to reduce the pressure differential between the reboiler section and the top of the column. The distillation columns are shown as packed columns in FIG. 2.

Depending upon the purity of the starting oleic acid and the amount of pelargonic acid recycled to reduce the viscosity of the ozonide in the ozonization/oxidation process, the amount of. pelargonic acid in the mixed oxidation products is in the range of from about 40 to about 70% by weight. A major portion of the compounds in the mixed oxidation product with boiling points higher than pelargonic acid along with some tarry materials are removed from the distillation column through line 35 and passed to a second distillation column 36. The azelaic acid and lower boiling point compounds in the mixture are separated from high boiling point residue in distillation column 36. The high boiling point residues leave distillation column 36 through line 37 and the prepurified azelaic acid leaves overhead of the distillation column 36 through line 38, is condensed into liquid in heat exchange means 39 and passed to dual extraction column 1 through line 2. A portion of the condensed overhead product is returned to column 36 as reflux through line 40.

The prepurified azelaic acid contains monocarboxylic acids with the same boiling range as azelaic acid along with C₈ to C₁₈ dicarboxylic acids. The prepurified azelaic acid enters the extractor 1 near the mid-point through line 2 and is immediately mixed and dispersed in the aqueous phase in contact with the water immiscible solvent. The water immiscible solvent generally has a lower specific gravity than the aqueous phase and flows upwardly against the downward flow of the aqueous phase through a multiplicity of mixing and packing zones.

In the upper portion of the column, the water immiscible solvent phase is contacted with an aqueous phase with a low content of azelaic acid and the aqueous phase extracts, from the water immiscible solvent phase, a portion of the azelaic acid which may be dissolved in the water immiscible solvent phase. In the lower portion of the column below the azelaic acid feed, the azelaic acid dissolved in the aqueous phase is contacted with the rising water immiscible solvent phase and the materials with a low water solubility are extracted into the water immiscible solvent phase.

The extractor 1 is preferably operated at a temperature in the range of from about 62° C. to about 95° C. The aqueous phase entering extractor 1 through line 5 and the water immiscible solvent phase entering extractor 1 through line 12 are pre-heated to a temperature in the range at which the column is operated. The aqueous phase entering extractor 1 through line 5 is introduced into the extractor at a point below a quiescent zone in which the water immiscible solvent phase separates from the aqueous phase. The water immiscible solvent phase leaves extractor 1 through line 8 passes to solvent recovery zone 18 in which the water immiscible solvent is separated from the materials which have been extracted from the prepurified azelaic acid and returned to extractor 1 through line 14, heat exchange means 13 and passes through line 12 to extractor 1 at a point above a quiescent zone in which the aqueous phase separates from the water immiscible solvent.

The aqueous phase leaves dual extractor 1 through line 11 and passes through evaporation-drying zone 20 in which the water is removed from the azelaic acid by evaporation and leaves zone 20 through line 21 and a molten azelaic acid passes from the evaporation-drying zone 20 through line 22 and enters distillation zone 23. In distillation zone 23, which is operated at an elevated bottom temperature ranging from about 235° C. to about 280° C. and a reduced pressure in the range of from about 1 mm Hg to about 30 mm Hg a portion of the azelaic acid feed with a boiling point lower than azelaic acid is removed as an overhead from the distillation zone 23 through line 24. Distillation zone 23 removes a portion of compounds which have a boiling point lower than that of azelaic acid, from the azelaic acid feed. Distillation zones 23 and 27 are preferably packed columns. Preferably the packing is of a low pressure drop type.

An azelaic acid with a reduced amount of lower boiling point materials is passed from distillation zone 23 through line 25 to second distillation zone 27. The second distillation zone 27 is operated at a temperature in a range of from about 220° C. to about 280° C. and a reduced pressure in the range of from about 1 mm Hg to about 10 mm of Hg. The purified azelaic acid is removed from distillation zone 27 through line 28 and a residue comprising higher boiling point materials is removed from distillation zone 27 through line 29. The purified azelaic acid which is removed from the distillation zone 27 is then passed to means for solidifying the azelaic acid into a commercial form. Generally, the azelaic acid is cooled and made in the form of flakes by a flaking roller or equivalent equipment. The high boiling point materials which contain polymers, tars, dicarboxylic acids with boiling points higher than azelaic acid and the color bodies can be treated to recover any valuable dicarboxylic acids

An azelaic acid product with good color and color stabibility can be prepared without ozonization of the aqueous solution of azelaic acid.

At times, azelaic acid and azelaic acid anhydrides form at the elevated temperatures in distillation zone 23. Anhydrides can be hydrolyzed by introducing a small amount of water into the azelaic acid product in line 25 through line 26. The mixture of the water with the azelaic acid product containing acid anhydrides on standing for a short period of time hydrolyses and returns the anhydride to the azelaic acid form. Alternatively, a small amount of water or steam can be introduced into the lower portion of distillation zone 27 to hydrolyze any anhydrides which may have been formed and are passing down through distillation zone 27 to be discarded with the high boiling point products in line 29. The introduction of water to reduce anhydride formation substantially increases the yield of azelaic acid from distillation zone 27. The purified azelaic acid is removed from distillation zone 27 through line 28.

The distillation conditions can be adjusted in distillation zone 23 and distillation zone 27 to produce an azelaic acid product with a required purity. That is, distillation parameters in distillation zone 23 can be adjusted to remove a larger portion of the lower boiling point components from the azelaic acid mixture. Conditions in distillation zone 27 can be adjusted to remove a larger proportion of the higher boiling point components to produce a more pure azelaic acid. However, the final distillation is generally utilized to remove any higher boiling point materials which may have formed during the extraction and first distillation procedure and/or to remove color causing materials which are usually high boiling point compounds. The dual extraction provides an azelaic acid with sufficiently low concentrations of acids which cannot be separated from azelaic acid by distillation that the azelaic acid can be recovered without a crystallization step.

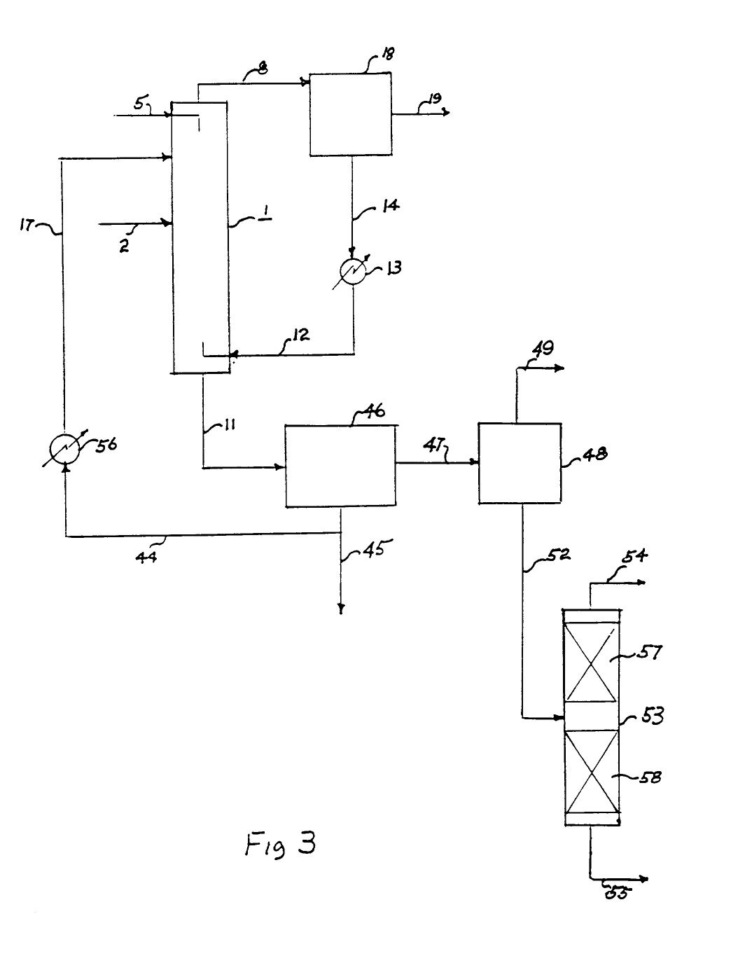

FIG. 3 is a diagrammatic representation of an embodiment of a process for recovering azelaic acid from a mixed oxidation product. In the process, prepurified azelaic acid is introduced into extraction zone 1 through line 2. The azelaic acid has been prepurified by the double distillation method disclosed in FIG. 2 which will not be repeated here. Hot water is introduced into the extractor through line 5 at a point below a separation zone for the water immiscible solvent which is introduced into the extraction zone through line 12. A heated aqueous recycle stream is introduced into extraction zone 1 through line 17. The recycle aqueous phase is introduced at a point 10-20 extraction stages below the top of extraction zone 1 and the prepurified azelaic acid is introduced near the mid-point of extraction zone 1 through line 2. A hot, water immiscible solvent for monocarboxylic acids with about 6 to 22 carbon atoms is removed from the extraction zone through line 8 and the water immiscible solvent is separated from the materials extracted from the azelaic acid in solvent recovery zone 18 and is recycled to extraction zone 1 through line 14, heat exchanger 13, and is introduced into extraction zone 1 through line 12. The aqueous phase containing dissolved purified azelaic acid is removed from extraction zone 1 through line 11 and is passed to crystallization zone 46.

In crystallization zone 46, the temperature of the aqueous phase is reduced, the azelaic acid crystallized and the crystals of azelaic acid separated from the aqueous phase. The aqueous phase is removed from the crystallization zone through line 44 and is heated by passing through heat exchange means 56 and is introduced into extraction zone 1 through line 17 at a point below the point where make-up water is introduced into the extraction zone. A portion of the aqueous liquid separated from the crystalline azelaic acid is removed through line 45 and passed to a treatment zone (not shown) in which the water soluble components can be recovered from the aqueous phase and if required the aqueous phase can be returned to the extraction zone.

It is necessary to remove a portion of the aqueous phase to prevent a build up of the soluble materials to a point at which it would not be possible to prevent them from crystallizing with the azelaic acid or dissolving additional compounds from the prepurified azelaic acid. The azelaic acid crystals are passed through conduit 47 to azelaic acid drying zone 48 wherein the moisture is removed from the azelaic acid through conduit 49 and a molten azelaic acid is passed through conduit 52 to distillation zone 53. In distillation zone 53, azelaic acid product is removed overhead through line 54 and high boiling point material containing color forming bodies is removed from the bottom of the distillation zone 53 through line 55. The distillation zone 53 preferably is a packed column with high efficiency low pressure drop packing.

The azelaic acid which is removed from the distillation zone is cooled and solidified. Generally, cooling and solidification is done on a flaking roller or an equivalent apparatus which provides for cooling of the azelaic acid and preparation of particulate azelaic acid. The high boiling point components are discarded or can be burned to reduce environmental pollution.

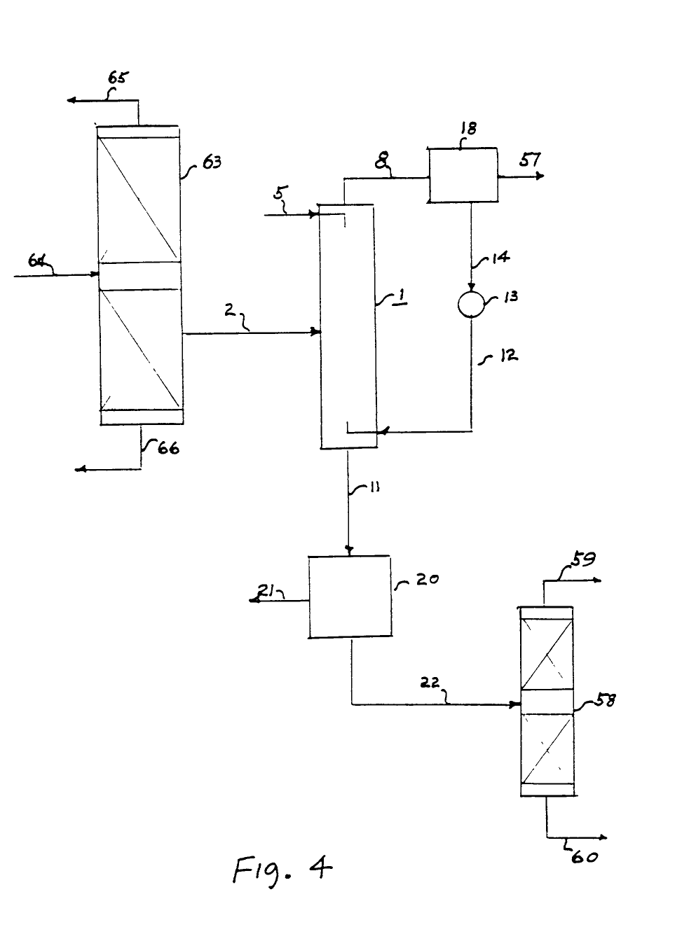

FIG. 4 is another illustration of a process utilizing the dual extraction zone of the present invention. A prepurified azelaic acid stream having a major portion of compounds with a boiling point lower than azelaic acid and compounds with boiling points higher than azelaic acid removed, is passed to a distillation zone 63 from a pre-purification zone such as in FIG. 2 (not shown). In distillation zone 63, a fraction of components with a boiling point lower than azelaic acid is removed as a distillate through line 65 and the residue portion with a boiling point higher than azelaic acid is removed from the bottom of distillation zone 63 through line 66. A side stream is removed from distillation zone 63 at a point below the feed point through line 2 and passed to the central portion of extraction zone 1. The side stream can be from the gas phase condensed or the liquid phase in column 63. Preferably the side stream is a condensed gas phase.

In extraction zone 1, the azelaic acid is contacted with a heated aqueous phase which is introduced into extraction zone 1 through line 5 and a heated water immiscible solvent for the impurities in the azelaic acid, introduced into the bottom portion of extraction zone 1 through line 12. The water immiscible solvent phase is removed as an overhead product from extraction zone 1 through line 8, is passed to solvent recovery zone 18, in which the solvent is separated from the materials extracted from the azelaic acid and the solvent returned to extraction zone 1 through line 14, heat exchange means 13 and line 12.

The materials extracted from the azelaic acid by the water immiscible solvent are removed from the system through line 57. An aqueous phase containing the dissolved azelaic acid which has substantially all of the monocarboxylic acids removed, is passed through line 11 to azelaic acid drying and melting zone 20 wherein the water is removed through line 21 by evaporation and the molten azelaic acid is passed through line 22 to distillation zone 58. In distillation zone 58 the azelaic acid is obtained as an overhead product through line 59 and a high boiling point residue is removed through line 60. If anhydride formation is a problem in distillation zone 58, a small amount of water can be introduced with the azelaic acid feed through line 22 or steam introduced into the bottom portion of distillation zone 58. Preferably distillation zones 63 and 58 are packed columns. Preferably the packing is of the high efficiency, low pressure drop type to reduce thermal effects on the materials.

One skilled in the art would quickly recognize that the azelaic acid purification process illustrated in FIG. 4 is related to the process disclosed in FIG. 2 with the first distillation column after the evaporation zone moved to a position ahead of the extraction zone to further purify the azelaic acid stream introduced into extraction zone 1.

FIGS. 1, 2, 3 and 4 are schematic representations only and do not include pumps, valves, temperature indicators, pressure indicators, vacuum producing means and other equipment which are generally associated with azelaic treatment zones illustrated in the figures. The process diagrams are schematic only and one skilled in the art would realize that control instrumentation, pumps, temperature indicators, pressure indicators and vacuum producing means would be necessary to make the process operable. However, to simplify the diagram, these various pieces of auxiliary equipment have not been shown.

Applicants have unexpectedly discovered that by use of the dual extraction zone in which the water immiscible solvent, for the water soluble materials, is extracted with an aqueous phase to recover a portion of azelaic acid dissolved in the water immiscible solvent and the water soluble materials dissolved in the aqueous phase are extracted from the aqueous phase containing the dissolved azelaic acid in a single extraction zone is an improvement over prior processes since the amount of azelaic acid lost in the water immiscible organic solvent is reduced and the operation can be carried out in a dual extraction zone without need for dissolving tanks for the azelaic acid and separation zones for the insoluble organic phase from the aqueous phase containing the dissolved azelaic acid.

It is preferred to utilize a single extraction zone. The extraction zone can be split into two or more continuously operated extracting zones. However, it is preferred that the extraction zone be a single extraction apparatus in which the aqueous feed is introduced near the top of the extractor and a water immiscible solvent with a specific gravity lower than the aqueous phase is introduced near the bottom of the extraction zone.

However, if a higher density water immiscible solvent is utilized, it can be introduced into the top of the apparatus and the aqueous phase introduced near the bottom.

The extraction zone is operated at a temperature in the range of from about 62 to about 95° C. and preferably in the range of from about 80 to about 90° C. to readily dissolve the azelaic acid and to be able to operate the extraction zone at substantially ambient pressure. Higher extraction temperatures can be utilized but require the use of pressurized equipment.

The dual extraction step of the present invention provides an azelaic acid which can be recovered from the aqueous phase without a crystallization step, the azelaic acid recovered by evaporation of the water from the aqueous phase can be further purified by mere distillation to provide an azelaic acid product containing more than 90% by weight of azelaic acid. The ability to provide an azelaic acid product containing more than 90% by weight of azelaic acid without a crystallization step has not been realized in the prior art. When a crystallization step is utilized with the dual extraction of the present invention, the aqueous phase from which the azelaic acid is crystallized can be recycled in part as a portion of the aqueous feed to the extraction zone. The ability to recycle the aqueous phase remaining after crystallization of the azelaic acid from the aqueous phase provides for a greater recovery of the azelaic acid and less problems of disposal of the aqueous phase. Up to about 85% by weight of the aqueous phase from the crystallization can be recycled as a portion of the aqueous phase to the extraction zone. Preferably, from about 60 to about 80% by weight of the aqueous phase is recycled. Since the azelaic acid is only soluble to a fixed amount in the aqueous phase after crystallization, and the amount of the aqueous phase which is disposed of is reduced by 60 to 80%, the amount of azelaic acid which is discarded with the other soluble materials is substantially reduced.

The distillation steps in the various processes disclosed are carried out at as low a temperature as is commercially feasible consistant with products being distilled. Generally, the distillation columns are operated at pressures in the range of 2 millimeters Hg to 100 millimeters of Hg and a temperature in the range of from about 150° C. to about 250° C. Operation of the distillation sections of the process is well known to one in the fatty acid arts, the specific conditions for each column will be dependent upon the overhead and under-flow products and the equipment available to produce the low pressures required.

The following examples are presented to illustrate operation of the dual extraction zone and the product obtainable by the process. The experiments were carried out in a 3-inch diameter by 11-feet, 4-inch-99 stage York-Scheibel column. The column was arranged as shown in FIG. 1 with the azelaic acid feed introduced near the center of the column and when recycle was utilized it was introduced at 12 stages from the top of the column. The column was operated at a temperature in the range of from about 76 to about 83° C. Pressure at the top of the column was ambient and the water immiscible solvent was VM&P Naphtha. All the process streams entering the column were heated to maintain the temperature in the extractor in the required temperature range.

Results of the experiments are shown in Table 1. Table 1 shows the analysis of the feed, and the non-volatile components of the solvent phase and the aqueous phase as they leave the column. The non-volatile components of the aqueous recycle are also shown in Table 1. The concentrations of the non-volatile components in the streams are not shown in the Table but they can be readily calculated from the flow rates and the compositions of the non-volatile matter set forth. In general, the extraction column was operated to maintain about a 13% by weight concentration of azelaic acid in the aqueous phase leaving the column. It can be seen from the data in Table 1 that it is possible to reduce the amount of monocarboxylic acids in the azelaic acid to levels less than 0.1% by weight. The azelaic acid content of the aqueous recycle stream was in the range of about 4% by weight.

TABLE 1

| | | | | | | | | | | | | | | | | | RECYCLE |
|---|---|---|---|---|---|---|---|---|---|---|---|---|---|---|---|---|---|
| | | | | | | | | | | | | | | | | | Aqueous |
| | | | | | | | | | | | | | | | | FEED | Phase |
| | | | | | | | | | | | | | | | | Compo- | Nonvolatile |
| | | | | | | | | | | | | | | | | sition | Composition |
| | SAMPLE | | | | | | | | | | | | | | | (weight | (weight |
| | A | B | C | O | D | E | F | G | H | I | J | K | L | M | N | percent) | percent) |
| | Feed Rate CC/Minute | | | | | | Feed Rate cc/Minute | | | | | | | | | | |
| Prepurified | 75 | 75 | 75 | 90 | 100 | 100 | 75 | 75 | 75 | 90 | 90 | 90 | 100 | 100 | 100 | — | — |
| Azelaic Acid | | | | | | | | | | | | | | | | | |
| VM & | 30 | 50 | 70 | 80 | 40 | 60 | 30 | 50 | 70 | 55 | 80 | 105 | 70 | 95 | 120 | — | — |
| P Naphtha | | | | | | | | | | | | | | | | | |
| Water | 425 | 425 | 425 | 510 | 560 | 560 | 110 | 110 | 110 | 130 | 130 | 130 | 145 | 145 | 145 | — | — |
| Recycle | — | — | — | — | — | — | 330 | 330 | 330 | 400 | 400 | 400 | 440 | 440 | 440 | — | — |
| | Solvent Phase | | | | | | Solvent Phase | | | | | | | | | | |
| | Nonvolatile Component | | | | | | Nonvolatile Component | | | | | | | | | | |
| Component | (weight Percent) | | | | | | (weight Percent) | | | | | | | | | | |
| <C8 Mono | 0.9 | 0.8 | 0.8 | 0.9 | 0.8 | 0.8 | 0.8 | 0.9 | 0.8 | 0.8 | 0.8 | 0.9 | 0.8 | 0.8 | 0.8 | 0.1 | — |
| C8-C12 Mono | 17.4 | 16.6 | 16.9 | 16.8 | 16.5 | 16.7 | 16.8 | 17.9 | 16.5 | 17.1 | 17.4 | 16.8 | 17.4 | 17.4 | 17.1 | 3.5 | — |
| C13-C16 Mono | 58.9 | 60.7 | 60.6 | 60.5 | 59.9 | 60.7 | 60.7 | 60.5 | 61.9 | 61.3 | 61.4 | 62.0 | 61.4 | 60.7 | 60.8 | 15.7 | — |
| >C16 Mono | 8.0 | 8.2 | 8.2 | 8.0 | 8.2 | 8.3 | 8.3 | 8.4 | 8.3 | 8.3 | 8.2 | 8.0 | 8.3 | 8.2 | 8.1 | 2.2 | — |
| <C8 DI | 0.0 | 0.0 | 0.0 | 0.1 | 0.0 | 0.1 | 0.2 | 0.2 | 0.2 | 0.2 | 0.2 | 0.5 | 0.2 | 0.2 | 0.2 | 3.8 | 59.3 |
| C8 DI | 0.2 | 0.2 | 0.2 | 0.2 | 0.2 | 0.2 | 0.3 | 0.3 | 0.3 | 0.3 | 0.3 | 0.4 | 0.4 | 0.4 | 0.4 | 3.1 | 21.1 |
| C9 DI | 4.3 | 4.2 | 4.4 | 5.0 | 4.3 | 4.3 | 4.9 | 5.1 | 4.9 | 5.0 | 5.1 | 5.2 | 5.0 | 5.3 | 5.6 | 61.2 | 17.5 |
| C10 DI | 0.4 | 0.3 | 0.3 | 0.3 | 0.5 | 0.4 | 0.7 | 0.6 | 0.6 | 0.6 | 0.6 | 0.6 | 0.6 | 0.7 | 0.6 | 2.5 | 1.4 |
| >C10 DI | 9.9 | 9.0 | 8.6 | 8.2 | 9.6 | 8.5 | 7.3 | 6.1 | 6.5 | 6.4 | 6.0 | 5.6 | 5.9 | 6.3 | 6.4 | 7.9 | 0.5 |

| | Aqueous Phase | | | | | | Aqueous Phase | | | | | | | | |
|---|---|---|---|---|---|---|---|---|---|---|---|---|---|---|---|
| | Nonvolatile Component | | | | | | Nonvolatile Component | | | | | | | | |
| Component | (Weight Percent) | | | | | | (weight Percent | | | | | | | | |
| <C8 DI | | 5.0 | | 5.1 | | 5.0 | | 14.6 | | | 12.2 | 12.9 | | 11.2 | |
| C8 DI | | 4.1 | | 4.0 | | 4.0 | | 6.9 | | | 6.0 | 6.1 | | 5.8 | |
| C9 DI | | 80.5 | | 80.5 | | 80.6 | | 68.8 | | | 72.5 | 71.6 | | 73.0 | |
| C10 DI | | 3.2 | | 3.1 | | 3.0 | | 2.7 | | | 2.6 | 2.6 | | 2.8 | |
| >C10 DI | | 7.2 | | 7.3 | | 7.4 | | 7.0 | | | 6.7 | 6.8 | | 7.2 | |
| Monobasic | 0.03 | 0.01 | 0.04 | 0.07 | 0.23 | 0.14 | 0.14 | 0.04 | 0.09 | 0.15 | 0.07 | 0.03 | 0.15 | 0.17 | 0.08 |
| Acid in the | | | | | | | | | | | | | | | |
| azelaic Acid | | | | | | | | | | | | | | | |
| Product After | | | | | | | | | | | | | | | |
| Distillation | | | | | | | | | | | | | | | |

We claim:

1. In a process in which azelaic acid is recovered from a mixture of carboxylic acids formed by ozonization and oxidation of a mixture comprising unsaturated fatty acids in the form of an oxidation mixture and wherein a portion of carboxylic acids with boiling points lower than azelaic acid are separated by a distillation procedure and a portion of the carboxylic acids with boiling points higher than azelaic acid are separated from the oxidation mixture to provide a prepurified azelaic acid mixture comprising monocarboxylic acids and dicarboxylic acids, the improvement which comprises continuously introducing the prepurified azelaic acid mixture into a dual extraction zone wherein the prepurified azelaic acid is dissolved in an aqueous phase and the aqueous phase is contacted with a water immiscible solvent phase for the monocarboxylic acids and the water immiscible solvent phase is contacted with the aqueous phase before the azelaic acid is dissolved in the aqueous phase to reduce the amount of water soluble material in the water immiscible solvent phase and recovering the azelaic acid from the aqueous phase.

2. The process of claim 1 wherein the prepurified azelaic acid mixture is extracted at a temperature in the range of from 62 to about 95° C.

3. The process of claim 1 wherein the ratio of aqueous phase to acid is sufficient to maintain the concentration of azelaic acid in the extracted aqueous phase in the range from about 10.5 to about 18% by weight.

4. The process of claim 1 wherein the weight ratio of water immiscible solvent to prepurified azelaic acid feed is in the range of from about 0.3:1 to about 2.5.

5. The process of claim 3 wherein the ratio of water immiscible solvent to prepurified azelaic acid feed is in the range of from about 0.4:1 to about 1.5:1.

6. The process of claim 1 wherein the water immiscible solvent is an aliphatic hydrocarbon composition having a boiling point of from about 90° C. to about 200° C.

7. The process of claim 1 wherein the azelaic acid is recovered from the aqueous phase by crystallization and the aqueous phase after crystallization of the azelaic acid is recycled to the dual extraction zone as a portion of the aqueous phase.

8. The process of claim 7 wherein the crystallized azelaic acid is further purified by a single distillation wherein the azelaic acid is recovered as a distillate and a high boiling fraction is removed.

9. A process of claim 1 wherein the aqueous phase containing the azelaic acid is dried and the azelaic acid melted, the molten azelaic acid is further purified in a two step distillation wherein in a first distillation step a top-cut is taken to remove carboxylic acids having a boiling point lower than that of azelaic acid from the azelaic acid and a second distillation step in which the azelaic acid is recovered as a distillate and a high boiling fraction is removed.

10. The process of claim 9 wherein water is introduced into the azelaic acid stream from the first distillation step to hydrolyze azelaic acid anhydride which may be present in the azelaic acid stream from the first distillation stage.

11. The process of claim 1 wherein the ratio by weight of the aqueous phase entering the dual extraction zone to the weight of prepurified azelaic acid is from about 3:1 to 9:1.

12. The process of claim 1 wherein the weight ratio of aqueous phase to water immiscible solvent phase is from about 3:5:1 to about 20:1 when the water immiscible solvent has a specific gravity of about 0.8 to about 0.9.

13. The process of claim 12 wherein the ratio is from about 4:1 to about 16:1.
